(19) United States
(12) Patent Application Publication
(10) Pub. No.: US 2008/0132568 A1
(43) Pub. Date: Jun. 5, 2008

(54) COMPOUNDS WITH ANTI-ANDROGENIC ACTIVITY AND THE USE THEREOF

(76) Inventors: Todd A. Thompson, Albuquerque, NM (US); Debra A. MacKenzie, Albuquerque, MN (US)

Correspondence Address: QUARLES & BRADY LLP, 411 E. WISCONSIN AVENUE, SUITE 2040, MILWAUKEE, WI, 53202-4497, US

(21) Appl. No.: 11/937,351

(22) Filed: Nov. 8, 2007

Related U.S. Application Data
(60) Provisional application No. 60/857,844, filed on Nov. 9, 2006.

(51) Int. Cl. A61K 31/277; A61K 31/185; A61K 31/13; A61K 31/135; A61K 31/165; A61K 31/12; A61K 31/095

(52) U.S. Cl. 514/520; 514/617; 514/553; 514/731; 514/641; 514/646; 514/706; 514/690

(57) ABSTRACT

Compounds having anti-androgenic activities are disclosed. Further disclosed are methods of using the compounds to prevent or treat androgen-dependent diseases or to provide nutraceutical benefits. Pharmaceutical and nutraceutical compositions containing the anti-androgenic compounds are also disclosed.

### CROSS-REFERENCE RELATED APPLICATION

This application claims the benefit of U.S. Patent Application No. 60/857,844, filed on Nov. 9, 2006, which is herein incorporated by reference in its entirety.

### STATEMENT REGARDING FEDERALLY SPONSORED RESEARCH OR DEVELOPMENT

Not applicable.

### BACKGROUND OF THE INVENTION

The major circulating steroids generally classified as androgens include dehydroepiandrosterone sulphate (DHEAS), dehydroepiandrosterone (DHEA), androstenedione, testosterone, and dihydrotestosterone (DHT) in descending order of serum concentration, though only the latter two bind the androgen receptor to a significant degree. The other three steroids are typically considered as pro-androgens. Testosterone is converted to its more active metabolite DHT by 5α-reductase. The action of androgens in the body is mediated by the androgen receptor (AR). Androgenic activity refers to the mediation of downstream effects such as gene expression modulation by the binding of an androgen (primarily DHT) to the AR.

Androgen-dependent diseases are well known in the art. Examples of such diseases include prostate cancer (including metastatic prostate cancer), benign prostatic hyperplasia, prostatic intraepithelial neoplasia, androgenic alopecia, hirsutism, acne, seborrhea, adrenal hyperplasia, precocious puberty, and polycystic ovary syndrome.

Androgen activity can be suppressed, for example, by androgen receptor antagonists, by reducing androgen biosynthesis, or by reducing the expression of the androgen receptor. These strategies can be applied to treat androgen-dependent diseases. For example, antiandrogens or androgen receptor antagonists such as flutamide (a nonsteroidal androgen receptor antagonist sold under the tradename Eulexin) have been used to treat androgen-dependent diseases such as prostate cancer, androgenic alopecia, and hirsutism.

### BRIEF SUMMARY OF THE INVENTION

It is disclosed here that the vitamin E oxidation product α-tocopherylquinone (ATQ) and certain derivatives thereof have anti-androgenic activity. Using ATQ and human prostate cancer cell lines as examples, the inventors have shown in the example below that ATQ inhibited the growth of androgen-dependent human prostate cancer cells as well as androgen-induced gene expression such as the expression of prostate specific antigen (PSA). In particular, ATQ and the derivatives thereof as anti-androgenic compounds are defined by formula I:

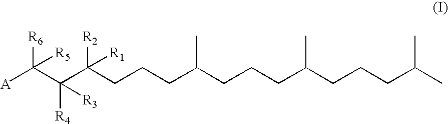

wherein A is structure II, III, or IV set forth below:

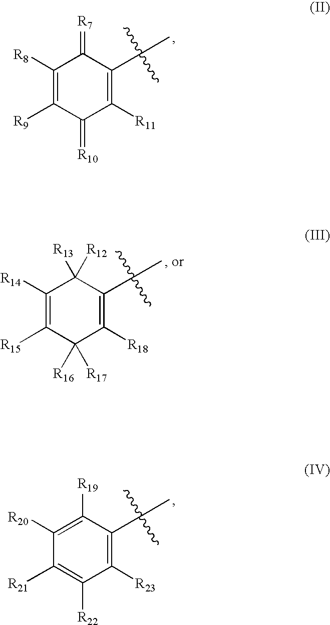

wherein R₁-R₆, R₈, R₉, and R₁₁-R₂₃ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₂ group, an alkylamino group defined as an —NHR group wherein R is an alkyl group having 1-3 hydrocarbons, a dialkylamino group defined as a —NRR′ group wherein R and R′ are alkyl groups having 1-3 hydrocarbons, an amido group defined as an —CONH₂ group, an alkylamido group defined as an —CONHR group wherein R is an alkyl group having 1-3 hydrocarbons, dialkylamido group defined as a —CONRR′ group wherein R and R′ are alkyl groups having 1-3 hydrocarbons, a nitro group defined as a —NO₂ group, a nitrile group defined as a —CN group, an acetate group defined as an —OAc group, a carbonyl group defined as a —CHO or —COR group wherein R is an alkyl group having 1-3 hydrocarbons, a carboxyl group defined as a —COOH group, an vinyl group defined as a —CH═CH₂ group, an ethinyl group defined as an —C≡CH group, a sulfonic acid group defined as a —SO₃H group, a sulfonamide group defined as a —SO₂NH₃ group, an alkylsulfonyl group defined as an —SO₂R group wherein R is an alkyl group having 1-3 hydrocarbons, a thio group defined as a —SH group, or a thioalkyl group defined as a —SR group wherein R is an alkyl group having 1-3 hydrocarbons, and

wherein R₇ and R₁₀ are identical or different and represent a methylene group defined as a ═CH₂ group, an ethylene group defined as an ═CHCH₃ group, an n-propylene group defined as a ═CHCH₂CH₃ group, an isopropylene group defined as an ═C(CH₃)₂ group, an imine group defined as an ═NH group, a thione group defined as a ═S bonded S, or an “one” group defined as an ═O bonded O.

Without intending to be limited by theory, the inventors believe that these compounds deliver anti-androgenic activity by down-regulating androgen receptor expression.

In one aspect, the present invention relates to a pharmaceutical composition comprising an anti-androgenic compound disclosed herein or a pharmaceutically acceptable salt or solvate thereof and a pharmaceutically acceptable carrier, with the proviso that the compound is not α-tocopherylquinone or α-tocopherylhydroquinone.

In another aspect, the present invention relates to a method for preventing or treating an androgen-dependent disease in a mammal such as a human or a dog. The method includes the step of administering to a mammal in need thereof a prophylactically or pharmaceutically effective amount of an anti-androgenic compound disclosed herein or a pharmaceutically acceptable salt or solvate thereof.

In another aspect, the present invention relates to a method for inhibiting the proliferation of androgen-dependent prostate tumor cells. The method includes the step of exposing the tumor cells to an anti-androgenic compound disclosed herein or a pharmaceutically acceptable salt or solvate thereof in an amount effective to inhibit the proliferation of the tumor cells.

In another aspect, the present invention relates to a nutraceutical composition comprising an anti-androgenic compound disclosed herein or a pharmaceutically acceptable salt or solvate thereof and an acceptable carrier, with the proviso that the compound is not α-tocopherylquinone or α-tocopherylhydroquinone.

In another aspect, the present invention relates to a method for providing nutraceutical benefits to a mammal such as a human or a dog. The method includes the step of administering to the mammal a nutraceutical composition that comprises an anti-androgenic compound disclosed herein or a pharmaceutically acceptable salt or solvate thereof.

### BRIEF DESCRIPTION OF THE SEVERAL VIEWS OF THE DRAWINGS

FIG. 1 shows the structure of vitamin E (VE), α-tocopherylquinone (ATQ), and α-tocopherylhydroquinone (ATHQ). The chromanol moiety of VE is oxidized by a 2 electron oxidation to produce ATQ, which can be reduced in the body by NADPH quinone oxidoreductase I (NQO1) to produce ATHQ (Siegel D et al., Molecular Pharmacology 1997, 52:300-305).

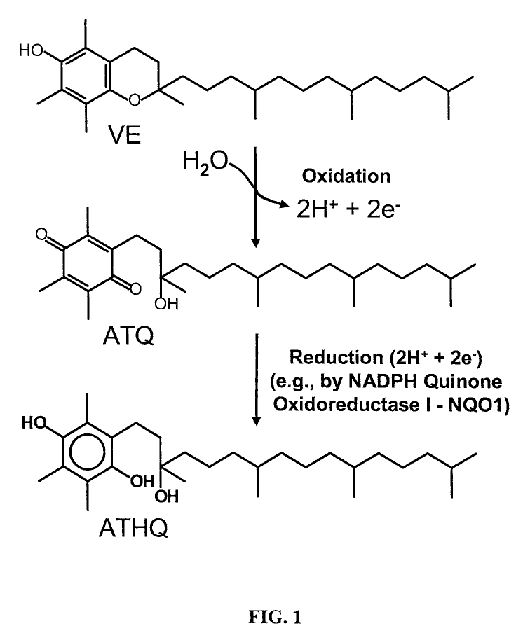

FIG. 2 shows the growth of prostate carcinoma cell lines after ATQ and VE treatment. A: LNCaP prostate cancer cells were exposed to either ATQ or VE for 4 days and the numbers of cells (i.e., relative growth) was determined using a CyQUANT assay. Cell growth was significantly decreased after treatment with 10 to 50 μM ATQ in LNCaP cells (P<0.05). Cell growth in LNCaP cells was not significantly altered by 10 to 50 μM VE treatment. B: LAPC4 prostate cancer cells were exposed to either ATQ or VE for 4 days and the number of cells was determined using a CyQUANT assay. Cell growth was significantly decreased after treatment with 10 to 50 μM ATQ (P<0.001). LAPC4 cell growth was decreased slightly by >30 μM VE treatment.

FIG. 3 shows androgen-induced promoter activity after ATQ and VE treatment in LNCaP prostate cancer cells. Androgen-induced (i.e., R1881) luciferase expression from an androgen-sensitive promoter was measured after ATQ or VE treatment for 48 hours. ATQ treatment significantly reduced androgen-sensitive promoter activity whereas VE treatment significantly increased promoter activity stimulated by exposure to 50 μM R1881; *(P<0.05).

FIG. 4 shows PSA levels in medium from LNCaP prostate cancer cells treated with ATQ or VE. PSA release was stimulated by 0.5 μM R1881 exposure and measured after 4 days of ATQ or VE treatment. PSA levels were significantly reduced after exposure to 10 or 40 μM ATQ (P<0.05), but remained unchanged after VE treatment.

FIG. 5 shows immunoblot analysis of AR protein levels in LNCaP cells (A) and LAPC4 cells (B) treated with ATQ and VE for 4 days. β-actin levels were used as a loading control.

FIG. 6 shows reduced levels of AR mRNA from LNCaP (A) and LAPC4 (B) cells 4 days after ATQ treatment (*P<0.05). VE did not affect AR mRNA levels as determined using qPCR (quantitative PCR).

### DETAILED DESCRIPTION OF THE INVENTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

In describing the embodiments and claiming the invention, the following terminology is used in accordance with the definitions set forth below.

As used herein the term “disease” is intended to include any abnormal physical or mental condition, including disorders, as well as any symptoms which are subject evidence of a disease or disorder.

As used herein, an “androgen-dependent disease” means a disease whose onset or progress is aided by the binding of an androgen to androgen receptor that mediates various downstream effects. These diseases are well known in the art and examples include, but are not limited to, prostate cancer (including metastatic prostate cancer), benign prostatic hyperplasia, prostatic intraepithelial neoplasia, androgenic alopecia (e.g., pattern baldness in men and women), hirsutism, acne, seborrhea, adrenal hyperplasia, precocious puberty, and polycystic ovary syndrome. For example, androgenic activity has been shown to be a contributing factor in the progression of prostate cancer, benign prostatic hyperplasia, androgenic alopecia, and hirsutism through, among other studies, the clinical evaluation of males who are genetically deficient of steroid 5α-reductase, the enzyme that produces dihydrotestosterone (DHT). With regard to hirsutism, facial and body hair growth are regulated by androgens, but opposite to that of the regulation of scalp hair. Specifically, DHT increases facial and body hair but reduces scalp hair. As another example, DHT has been shown to increase the production of sebum (oil) from the sebaceous gland, which causes seborrhea and contributes to an increase in acne.

As used herein, the terms “reducing” and “inhibiting” have their commonly understood meaning of lessening or decreasing. As used herein, the term “progression” means increasing in scope or severity, advancing, growing or becoming worse. As used herein, the term “recurrence” means the return of a disease after a remission.

By “treating” a disease, we mean achieving a therapeutic benefit which includes eradicating or ameliorating the underlying disorder, eradicating, ameliorating, or reducing one or more of the pathophysiological symptoms associated with the underlying disorder, or preventing or slowing down the progression of the disease.

By “preventing” a disease, which includes preventing the recurrence of a disease, we mean preventing the development of the disease, delaying the development of the disease, or reducing the severity of the disease at the onset of the disease. For example, a compound according to the present invention can be administered to a patient at risk of developing an androgen-dependent disease or a patient reporting one or more of the pathophysiological symptoms of an androgen-dependent disease even though a diagnosis may not have been made.

As used herein, a “therapeutically effective amount” means the total amount of each active component of the pharmaceutical composition or method that is sufficient to treat a disease as defined above. As used herein, a “prophylactically effective amount” means the total amount of each active component of the pharmaceutical composition or method that is sufficient to prevent a disease as defined above. When applied to an individual active component, administered alone, the terms refer to that component alone. When applied to a combination, the terms refer to combined amounts of the active components that result in the therapeutic or prophylactic effect, whether administered in combination, concurrently or sequentially. The specific “effective amount” will vary with such factors as the particular disease being treated, the physical condition of a patient being treated, the type of mammal being treated, the duration of the treatment, the nature of concurrent therapy (if any), and the specific formulations employed as well as the structure of the compounds. The optimum effective amounts can be readily determined by one of ordinary skill in the art using routine experimentation. The total daily dosage may be divided and administered in portions during the day.

The present invention provides a new class of compounds with anti-androgenic activities as well as methods of using the compounds for preventing or treating androgen-dependent diseases. The compounds can also be provided in a nutraceutical composition to achieve nutraceutical benefits. This class of anti-androgenic compounds is defined by formula I:

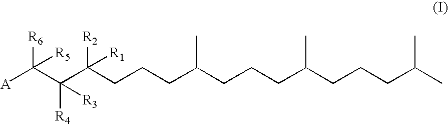

wherein A is structure II, III, or IV set forth below:

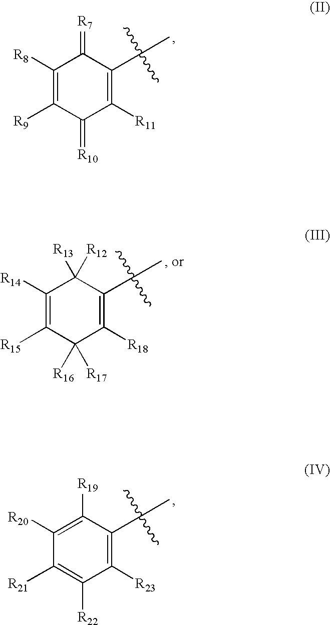

wherein R₁-R₆, R₈, R₉, and R₁₁-R₂₃ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₂ group, an alkylamino group defined as an —NHR group wherein R is an alkyl group having 1-3 hydrocarbons, a dialkylamino group defined as a —NRR′ group wherein R and R′ are alkyl groups having 1-3 hydrocarbons, an amido group defined as an —CONH₂ group, an alkylamido group defined as an —CONHR group wherein R is an alkyl group having 1-3 hydrocarbons, dialkylamido group defined as a —CONRR′ group wherein R and R′ are alkyl groups having 1-3 hydrocarbons, a nitro group defined as a —NO₂ group, a nitrile group defined as a —CN group, an acetate group defined as an —OAc group, a carbonyl group defined as a —CHO or —COR group wherein R is an alkyl group having 1-3 hydrocarbons, a carboxyl group defined as a —COOH group, an vinyl group defined as a —CH═CH₂ group, an ethinyl group defined as an —C≡CH group, a sulfonic acid group defined as a —SO₃H group, a sulfonamide group defined as a —SO₂NH₃ group, an alkylsulfonyl group defined as an —SO₂R group wherein R is an alkyl group having 1-3 hydrocarbons, a thio group defined as a —SH group, or a thioalkyl group defined as a —SR group wherein R is an alkyl group having 1-3 hydrocarbons, and

wherein R₇ and R₁₀ are identical or different and represent a methylene group defined as a ═CH₂ group, an ethylene group defined as an ═CHCH₃ group, an n-propylene group defined as a ═CHCH₂CH₃ group, an isopropylene group defined as an ═C(CH₃)₂ group, an imine group defined as an ═NH group, a thione group defined as a ═S bonded S, or an “one” group defined as an ═O bonded O.

By an “alkyl group having 1-3 hydrocarbons,” we mean —CH₃, —CH₂CH₃, —CH₂CH₂CH₃ or —CH(CH₃)₂.

In one embodiment, A is structure II. In one form of this embodiment, R₇ and R₁₀ are identical or different and represent an imine group defined as an ═NH group, a thione group defined as a ═S bonded S, or an “one” group defined as an ═O bonded O.

In another embodiment, A is III. In one form of this embodiment, R₁₂, R₁₃, R₁₆, and R₁₇ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₂ group, an alkylamino group defined as an —NHR group wherein R is an alkyl group having 1-3 hydrocarbons, a dialkylamino group defined as a —NRR′ group wherein R and R′ are alkyl groups having 1-3 hydrocarbons, a nitro group defined as a —NO₂ group, a nitrile group defined as a —CN group, a carbonyl group defined as a —CHO or —COR group wherein R is an alkyl group having 1-3 hydrocarbons, a carboxyl group defined as a —COOH group, an vinyl group defined as a —CH═CH₂ group, an ethinyl group defined as an —C≡CH group, a sulfonic acid group defined as a —SO₃H group, an alkylsulfonyl group defined as an —SO₂R group wherein R is an alkyl group having 1-3 hydrocarbons, a thio group defined as a —SH group, or a thioalkyl group defined as a —SR group wherein R is an alkyl group having 1-3 hydrocarbons.

In another embodiment, A is IV. In one form of this embodiment, R₁₉, and R₂₂ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₂ group, an alkylamino group defined as an —NHR group wherein R is an alkyl group having 1-3 hydrocarbons, a thio group defined as a —SH group, or a thioalkyl group defined as a —SR group wherein R is an alkyl group having 1-3 hydrocarbons.

In one preferred embodiment, the anti-androgenic compounds are defined by formula I wherein A is structure II or IV, wherein R₁ and R₂ are identical or different and represent a hydrogen defined as a —H atom, a methyl group defined as a —CH₃ group, or a hydroxyl group defined as a —OH group, wherein R₃, R₄, R₅, and R₆ are identical or different and represent a hydrogen defined as a —H atom or a methyl group defined as a —CH₃ group, wherein each of R₈, R₉, R₁₁, R₂₀, R₂₁, and R₂₃ is a methyl group defined as a —CH₃ group, wherein R₇ and R₁₀ are identical or different and represent a thione group defined as a ═S bonded S or an “one” group defined as an ═O bonded O, and wherein R₁₉ and R₂₂ are identical or different and represent a hydroxyl group defined as a —OH group or a thio group defined as a —SH group.

In one form of the preferred embodiment, the compound defined by formula I is α-tocopherylquinone (ATQ). In another form of the preferred embodiment, the compound defined by formula I is α-tocopherylhydroquinone (ATHQ).

As one of ordinary skill in the art appreciates, some compounds of formula I can form salts and these salts are also within the scope of the present invention. Reference to a compound of formula I herein is understood to include reference to salts thereof when applicable, unless otherwise indicated. The term “salt(s),” as employed herein, denotes acidic salts formed with inorganic and/or organic acids, as well as basic salts formed with inorganic and/or organic bases. Pharmaceutically acceptable (i.e., non-toxic, physiologically acceptable) salts are preferred, although other salts are also useful.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

Compounds of formula I, and salts and solvates thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts and solvates of the compounds), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” and the like is intended to equally apply to the salt and solvate of enantiomers, stereoisomers, rotamers, tautomers, positional isomers, or racemates of the anti-androgenic compounds disclosed herein.

In one aspect, the present invention relates to a pharmaceutical composition comprising an anti-androgenic compound disclosed herein or a pharmaceutically acceptable salt or solvate thereof and a pharmaceutically acceptable carrier, with the proviso that the compound is not α-tocopherylquinone or α-tocopherylhydroquinone.

In another aspect, the present invention relates to a method for preventing or treating an androgen-dependent disease in a mammal such as a human or a dog. The method includes the step of administering to a mammal in need thereof a prophylactically or pharmaceutically effective amount of an anti-androgenic compound disclosed herein or a pharmaceutically acceptable salt or solvate thereof. For example, ATQ or a pharmaceutically acceptable salt or solvate thereof may be administered.

In one embodiment, the method is for preventing an androgen-dependent disease. In another embodiment, the method is for treating an androgen-dependent disease. In another embodiment, the method is for preventing prostate cancer. In another embodiment, the method is for treating prostate cancer. In another embodiment, the method is for preventing benign prostatic hyperplasia. In another embodiment, the method is for treating benign prostatic hyperplasia. In another embodiment, the method is for preventing androgenic alopecia such as male or female androgenic alopecia. In another embodiment, the method is for treating androgenic alopecia such as male or female androgenic alopecia.

The method disclosed above can optionally include a step of evaluating the effectiveness of the treatment in connection with treating an androgen-dependent disease. This can be done, for example, by monitoring the disease itself or one or more symptoms of the disease. In the case of treating prostate cancer, the effectiveness of the treatment can be determined by, for example, monitoring the size of the prostate tumor (may be monitored by, e.g., ultrasound or magnetic resonance imaging). A slow down in tumor size increase, a stabilization in tumor size, or a decrease in the size of the tumor indicates that the treatment is effective. Similarly, for benign prostatic hyperplasia, the effectiveness of the treatment can be determined by, for example, monitoring the size of the prostate gland. A slow down in gland size increase, a stabilization in gland size, or a decrease in the size of the gland indicates that the treatment is effective. For andogenic alcopecia, the effective of the treatment can be observed, for example, by hair regrowth or a decrease in the rate of hair loss, preferably by a cosmetically significant amount.

While an anti-androgenic compound disclosed herein can be administered as the sole active ingredient for preventing or treating an androgen-dependent disease, more than one anti-androgenic compounds disclosed herein can be administered either in combination, concurrently or sequentially. Further, one or more of the anti-androgenic compounds disclosed herein can be administered with other agents such as other anti-androgenic compounds (e.g., androgen antagonists and 5-α reductase inhibitors) that are effective for preventing or treating the disease. In the case of treating prostate cancer, one or more anti-androgenic compounds disclosed herein can be administered with another anti-cancer drug such as a chemotherapeutic agent (e.g., alkylating agents, antimetabolites, natural products and their derivatives, hormones and steroids, and synthetics).

As used herein, a “pharmaceutical composition” means therapeutically or prophylactically effective amount of an anti-androgenic compound together with a pharmaceutically acceptable carrier. Examples of pharmaceutically acceptable carriers can be found in Remington's Pharmaceutical Sciences, 19th edition, A. R. Gennaro, Ed., Mack Publ., Easton, Pa. (1995).

The present invention is not limited to any particular route of administration and any suitable route of administration can be employed. For example, a pharmaceutical composition according to the present invention can be administered orally, parenterally (e.g., subcutaneously, intradermally, intramuscularly, intraperitoneally, and intravenously), topically, or transdermally. In one embodiment, a pharmaceutical composition is administered orally, topically, or intravenously. For treating prostate cancer and benign prostatic hyperplasia, the pharmaceutical composition may be administered to the tumor or hyperplastic tissue directly (e.g., intratumorally) or to a region in proximity to the tumor of hyperplastic tissue (e.g., paracancerally). For treating androgenic alopecia, a pharmaceutical composition according to the present invention may be applied to the scalp directly.

The pharmaceutical compositions may contain formulation materials for modifying, maintaining or preserving, for example, the pH, osmolarity, oxidation state, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as tert-butylhydroquinone, butylated hydroxyanisole, butylated hydroxytoluene, ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents [such as ethylenediamine tetra-acetic acid (EDTA)]; complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides); delivery vehicles; diluents; excipients and/or pharmaceutical adjuvants. See Remington's Pharmaceutical Sciences, 19th edition, A. R. Gennaro, Ed., Mack Publ., Easton, Pa. (1995).

The optimal pharmaceutical composition will be determined by one skilled in the art depending upon, for example, the intended route of administration, delivery format, and desired dosage. See, for example, Remington's Pharmaceutical Sciences, supra. Such compositions may influence the physical state, stability, rate of in vivo release, and rate of in vivo clearance of the anti-androgenic compounds disclosed herein.

The primary vehicle or carrier in a pharmaceutical composition may be either aqueous or non-aqueous in nature. For example, a suitable vehicle or carrier may be water or physiological saline solution, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present invention, the pharmaceutical compositions may be prepared for storage by mixing the selected composition having the desired degree of purity with optional formulation agents (Remington's Pharmaceutical Sciences, supra) in the form of a lyophilized cake or an aqueous solution. Further, the pharmaceutical composition may be formulated as a lyophilizate using appropriate excipients such as sucrose.

When oral administration is contemplated, the anti-androgenic compounds can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the anti-androgenic compounds. Diluents, flavorings, low melting point waxes, vegetable oils, lubricants, suspending agents, tablet disintegrating agents, and binders may also be employed.

Pharmaceutical compositions for oral administration can also be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by a patient.

Pharmaceutical compositions for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical compositions that can be used orally also include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Another pharmaceutical composition may involve an effective quantity of an anti-androgenic compound disclosed herein in a mixture with non-toxic excipients that are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

When parenteral administration is contemplated, the pharmaceutical compositions for use in this invention may be in the form of a pyrogen-free, parenterally acceptable aqueous solution comprising one or more anti-androgenic compounds disclosed herein in a pharmaceutically acceptable vehicle. A particularly suitable vehicle for parenteral injection is sterile distilled water in which an anti-androgenic compound is formulated as a sterile, isotonic solution, properly preserved. Yet another preparation can involve the formulation of an anti-androgenic compound with an agent, such as injectable microspheres, bio-erodible particles, polymeric compounds (polylactic acid, polyglycolic acid), beads, or liposomes that provides for the controlled or sustained release of the product which may then be delivered via a depot injection. Hyaluronic acid may also be used, and this may have the effect of promoting sustained duration in the circulation. Other suitable means for the introduction of an anti-androgenic compound include implantable drug delivery devices.

In another embodiment, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

For topical administration to body surfaces using, for example, creams, gels, drops, and the like, the anti-androgenic compounds disclosed herein may be prepared and applied as solutions, suspensions, or emulsions in a physiologically acceptable diluent.

Additional pharmaceutical compositions will be evident to those skilled in the art, including formulations involving anti-androgenic compounds disclosed herein in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-release preparations include semipermeable polymer matrices in the form of shaped articles, e.g., films or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate, or poly-D(−)-3-hydroxybutyric acid. Sustained-release compositions also include liposomes, which can be prepared by any of several methods known in the art.

The pharmaceutical composition to be used for in vivo administration may need to be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In another aspect, the present invention relates to a method for inhibiting the proliferation of androgen-dependent prostate tumor cells. The method includes the step of exposing the tumor cells to an anti-androgenic compound disclosed herein or a pharmaceutically acceptable salt or solvate thereof in an amount effective to inhibit the proliferation of the tumor cells.

In addition, one or more anti-androgenic compounds disclosed herein may be provided in the form of a nutraceutical composition where the anti-androgenic compounds prevent the onset of or reduce or stabilize various androgen-dependent diseases, e.g., prostate cancer. In one embodiment, such a nutracentical composition comprises an anti-androgenic compound or a pharmaceutically acceptable salt or solvate thereof provided herein with the proviso that the compound is not α-tocopherylquinone or α-tocopherylhydroquinone. The term “nutraceutical composition,” for the purposes of this specification, refers to a food item, a part of a food item, or a dietary supplement, that contains a natural or synthesized substance which offers medical health benefits, including prevention and/or treatment of disease. A nutraceutical composition according to the present invention may contain only an anti-androgenic compound according to the present invention as an active ingredient or, alternatively, may further contain, in admixture with the aforesaid anti-androgenic compound, dietary supplements such as vitamins, co-enzymes, minerals, herbs, amino acids and the like.

Therefore, the present invention provides methods of providing nutraceutical benefits (e.g., preventing or ameliorating an androgen-dependent disease) to a mammal such as a human or a dog including the step of administering to the mammal a nutraceutical composition containing a compound defined by formula I or a pharmaceutically acceptable salt or solvate thereof. Such compositions generally include a “nutraceutically acceptable carrier” which, as referred to herein, is any carrier suitable for oral delivery including, but not limited to, the aforementioned pharmaceutically acceptable carriers. In certain embodiments, nutraceutical compositions according to the invention further comprise dietary supplements which, defined on a functional basis, include immune boosting agents, anti-viral agents, anti-oxidant agents, anti-inflammatory agents, or mixtures thereof.

The immune boosters and anti-viral agents are useful for improving immune function and they include extracts from the coneflowers or herbs of the genus Echinacea, extracts from herbs of the genus Sambuca, and Goldenseal extracts. Herbs of the genus Astragalus are also effective immune boosters in either their natural or processed forms. Astragalus stimulates development of stem cells in the marrow and lymph tissue into active immune cells. Zinc and its bioactive salts, such as zinc gluconate and zinc acetate, also act as immune boosters in the treatment of the common cold.

Antioxidants include the natural, sulfur-containing amino acid allicin, which acts to increase the level of antioxidant enzymes in the blood. Herbs or herbal extracts, such as garlic, which contain allicin are also effective antioxidants. The catechins and the extracts of herbs such as green tea containing catechins are also effective antioxidants. Extracts of the genus Astragalus also show antioxidant activity. The bioflavonoids, such as quercetin, hesperidin, rutin, and mixtures thereof, are also effective as antioxidants. The primary beneficial role of the bioflavonoids may be in protecting vitamin C from oxidation in the body. This makes more vitamin C, or ascorbic acid, available for use by the body.

Bioflavonoids such as quercetin are also effective anti-inflammatory agents, and may be used as such in the inventive compositions. Anti-inflammatory herbal supplements and anti-inflammatory compounds derived from plants or herbs may also be used as anti-inflammatory agents. These include bromolain (a proteolytic enzyme found in pineapple), teas, extracts of stinging nettle, turmeric, extracts of turmeric, and curcumin (a yellow pigment isolated from turmeric).

Supplements useful in treating migraine headaches include feverfew and Gingko biloba. The main active ingredient in feverfew is the sesquiterpene lactone parthenolide, which inhibits the secretion of prostaglandins which in turn cause pain through vasospastic activity in the blood vessels. Feverfew also exhibits anti-inflammatory properties. Fish oil, owing to its platelet-stabilizing and antivasospastic actions, may also be useful in treating migraine headaches. The herb Gingko biloba also assists in treatment of migraines by stabilizing arteries and improving blood circulation.

Another supplement which may be used in the present invention is ginger, derived from herbs of the genus Zingiber. This has been found to possess cardiotonic activity due to compounds such as gingerol and the related compound shogaol as well as providing benefits in the treatment of dizziness and vestibular disorders. Ginger is also effective in the treatment of nausea and other stomach disorders.

Supplements which assist in rebuilding soft tissue structures, particularly in rebuilding cartilage, are useful in compositions for treating the pain of arthritis and other joint disorders. Glucosamine, glucosamine sulfate, chondroitin, and chondroitin sulfate are particularly useful for this purpose. Chondroitin may be derived from a variety of sources, such as Elk Velvet Antler. Marine lipid complexes, omega 3 fatty acid complexes, and fish oil are also known to be useful in treating pain associated with arthritis.

By way of example, but not limitation, an example of the present invention is described below.

### EXAMPLE

### α-tocopherylquinone and vitamin E Differentially Modulate Growth, Viability, and Androgen-Induced Gene Expression in Human Prostate Cancer Cells

The major oxidation product of vitamin E (VE) is α-tocopherylquinone (ATQ), which is chemically distinct from VE. In this example, ATQ and VE were compared for their effects on prostate cancer cell growth, cell death, and androgen-induced gene expression in androgen-sensitive human prostate carcinoma cell lines. ATQ treatment produced a dose-dependent decrease in the growth of both LAPC4 and LNCaP human prostate carcinoma cells. In contrast, treatment with VE was much less effective in altering LAPC4 and LNCaP growth over 4 days. Because LAPC4 and LNCaP human prostate cancer cells are androgen-sensitive, the ATQ-mediated effects on androgen responsiveness in these cell lines were compared to VE. ATQ was found to inhibit androgen-induced expression from androgen-driven reporter vectors. Surprisingly, and in contrast to ATQ, VE increased androgen-induced expression from androgen-driven reporter vectors. ATQ also inhibited the androgen-induced expression of prostate specific antigen (PSA) from LNCaP cells. In contrast, VE treatment did not affect PSA release. The changes observed in androgenic responses by ATQ may be due to modulation of androgen receptor protein levels which were decreased in both ATQ-treated LAPC4 and LNCaP cells. VE did not affect androgen-receptor protein levels. Furthermore, a 2-fold decrease in androgen receptor mRNA was observed after ATQ treatment. Thus, ATQ, but not VE, was found to decrease growth, viability, and androgen-induced gene expression of human prostate cancer cells.

Abbreviations Used in this Example

AR, androgen receptor; ATQ, α-tocopherylquinone; CSS, charcoal-stripped serum; FCS, fetal calf serum; GAPDH, glyceraldehyde phosphate dehydrogenase; MMTV, mouse mammary tumor virus; PCR, polymerase chain reaction; PSA, prostate specific antigen; VE, vitamin E; dsEGFP, destabilized enhanced green fluorescence protein.

Materials and Methods

Chemicals: α-tocopherylquinone as dl-ATQ was obtained from Research Organics (Cleveland, Ohio). Methyltrienolone (i.e., R1881) was obtained from Perkin Elmer/NEN Life Science Products (Boston, Mass.). VE as dl-α-tocopherol and other chemicals used were purchased from Sigma Chemical Co (St. Louis, Mo.).

Cell culture and treatment protocols: The LNCaP prostate cancer cells used in these studies were acquired from American Type Culture Collection (Manassas, Va.). All cell lines were maintained in Dulbecco's modified Eagle's medium (DMEM; Invitrogen, Carlsbad, Calif.) containing 5% heat-inactivated fetal calf serum (FCS; Sigma, St. Louis, Mo.) with streptomycin-penicillin antibiotics (designated DMEM/FCS) in a 5% CO₂ incubator at 37° C. LAPC4 prostate cancer cells adapted to growth in DMEM and 5% FCS were acquired from George Wilding (University of Wisconsin Paul P. Carbone Comprehensive Cancer Center). For experiments evaluating androgenic responses, cells were cultured in DMEM containing 4% charcoal-stripped FCS and 1% unstripped FCS (designated DMEM/CSS). To treat cells with 25 μM VE or 25 μM ATQ, 100 μL of VE or ATQ at 5 mM in 100% ethanol was added to 900 μL of 7.5% bovine albumin fraction V (Invitrogen) at 37° C. with continuous mixing. This was added to 19 mL of DMEM/CSS for cell treatment. To treat cells with 30 μM VE or 30 μM ATQ, 100 μL of VE or ATQ at 6 mM in 100% ethanol was added to 900 μL of 7.5% bovine albumin fraction V (Invitrogen) at 37° C. with continuous mixing. This was added to 19 mL of DMEM/CSS for cell treatment.

Cell proliferation and viability assays: Cell numbers were determined using a hemacytometer. Relative cell growth changes were determined using cells plated in 96-well tissue culture plates using the CyQUANT NF Cell Proliferation Assay Kit (Invitrogen) according to manufacturer's instructions. Cell viability was measured using trypan blue exclusion and quantified using light microscopy and hemacytometry. Additionally, cell viability was measured by propidium iodide exclusion using flow cytometry (FACScan; BD Biosciences, San Jose, Calif.) after VE and ATQ treatment.

Immunoblot analysis: LNCaP or LAPC4 prostate cancer cells were plated at a density of 1×10⁶ cells per 100 mm cell culture plate in 10 ml of DMEM/CSS and maintained in incubators at 37° C. in 5% CO₂. After 4 days of treatment with vehicle, 25 or 30 μM VE, or 25 or 30 μM ATQ, cells were washed in cold 1×PBS and lysed in a buffer containing 1.0% Igepal CA-630, 0.5% sodium deoxycholate, 0.1% sodium dodecyl sulfate, 0.1 mg/ml phenylmethylsulfonyl fluoride, 1 mM sodium orthovanadate, and 10 μg/ml aprotinin in 1×PBS. Cell extracts were stored at −80° C. until analysis. Total protein (25 μg) from cell extracts were electrophoresed on 7.5% SDS-polyacrylamide gels and transferred to Immobilon-P membranes (Millipore Corp., Bedford, Mass.) using a GENIE wet transfer system (Idea Scientific, Minneapolis, Minn.). Membranes were blocked in Tris-buffered saline containing 5% nonfat dry milk and then incubated with mouse anti-AR (441) monoclonal antibody (Santa Cruz Biotechnology, Santa Cruz, Calif.) or mouse anti-actin antibody (A5441; Sigma). After washing, membranes were incubated with a secondary horseradish peroxidase-conjugated goat anti-mouse IgG (Biomeda, Foster City, Calif.) and analyzed using Western Lightening Chemiluminescence Reagent Plus (Boston, Mass.) on a Kodak Image Station 4000MM (Rochester, N.Y.). Band intensities were determined using Kodak Molecular Imaging Software.

Messenger RNA expression analysis: Total RNA was extracted from cells using TRIzol Reagent (Invitrogen, Carlsbad, Calif.) and cDNA was prepared from total RNA using the High Capacity cDNA Reverse Transcription Kit (Applied Biosystems, Foster City, Calif.). Quantititive PCR was performed for AR expression using an Applied Biosystems 7900HT Fast Real-Time PCR System (Carlsbad, Calif.) and QuantiTect Primers Assays (Qiagen Inc., Valencia, Calif.) for AR and GAPDH mRNA.

Prostate specific antigen analysis: LNCaP cells were cultured in 96-well plates (Costar) at 5,000 cells per well in DMEM/CSS 1 day before treatment. Forty-eight hours after treatment, media levels of PSA released from LNCaP cells were measured using a PSA Enzyme Immunoassay Test Kit (BioCheck, Inc., Foster City, Calif.) according to manufacturer's instructions supplied with the kit. PSA levels were normalized to cell numbers as determined using the CyQUANT NF Cell Proliferation Assay Kit (Invitrogen), as described previously.

Promoter activation assay: LNCaP and LAPC4 prostate carcinoma cell lines were cultured in 12- or 24-well cell culture plates (Costar) in DMEM/CSS 2 to 3 days before transfection. Androgen-induced transcriptional activation was determined using a reporter construct with an MMTV promoter that regulates the expression of luciferase (Thompson T A et al., In Vitro Cell. Dev. Biol. 1993, 29A:165-170). LNCaP cells were transfected using the calcium phosphate precipitation method with an MMTV/luciferase plasmid. LAPC4 cells were transfected with an MMTV/luciferase plasmid using the FuGENE 6 Transfection Reagent (Roche Applied Science), according to the manufacturer's instructions. Twenty-four hours after transfection, cells were treated with R1881 with or without test reagents at the specified concentrations. Cell extracts were acquired 24 to 48 hours after treatment in 100 μL of Cell Culture Lysis Reagent (Promega, Madison, Wis.). Luciferase activity was measured using the Luciferase Assay Substrate (Promega, Madison, Wis.) according to instructions and read on a TD-20/20 Luminometer (Turner Designs, Sunnyvale, Calif.).

Statistical analysis. Significant differences in values between groups were assessed using an unpaired t-test with SigmaStat 3.1 software (Systat Software, Inc., San Jose, Calif.). P values less than 0.05 were used to signify statistical significance.

### Results

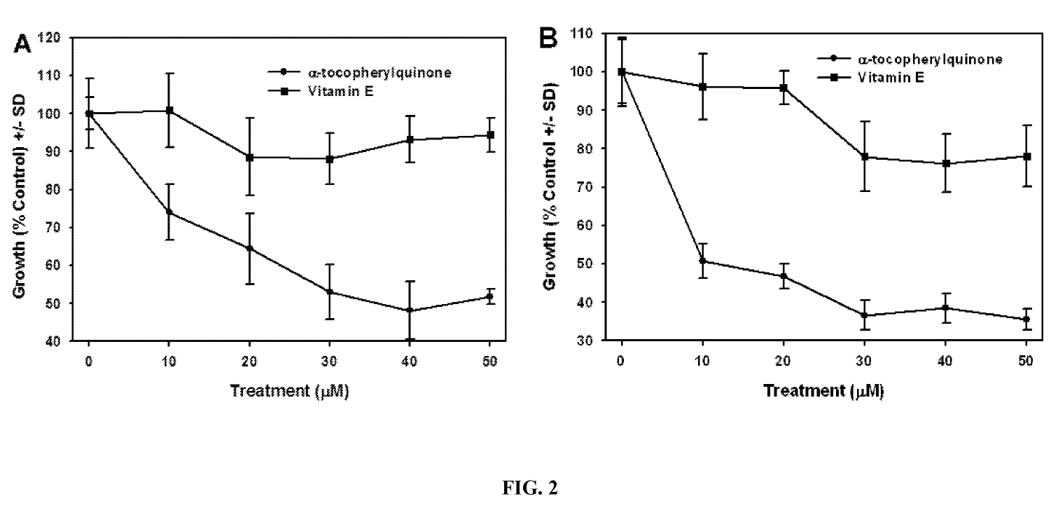

Prostate cancer cell growth inhibition by ATQ treatment: Treatment with ATQ produced a dose-dependent decrease in prostate cancer cell growth over a 4 day period in LNCaP and LAPC4 androgen-sensitive prostate cancer cells, which was significant at the lowest 10 μM dose of ATQ (FIG. 2). In contrast, VE did not significantly decrease the growth of LNCaP cells in treatments up to 50 μM (FIG. 2A). A modest, but significant, decrease in LAPC4 cell growth was observed at VE treatment levels equal to or greater than 30 μM. In contrast, neither ATQ nor VE decreased the growth of the androgen-independent PC3 prostate cancer cell line after treatment for 4 days (data not shown). For LNCaP and LAPC4 cells, a physiologically relevant 20 to 30 μM dose of ATQ and VE allowed the distinction of selective ATQ responses. Therefore, a 20 to 30 μM dose of ATQ and VE was used for most of the studies performed.

Prostate cancer cell death after ATQ treatment: Cell viability was significantly decreased in LNCaP cells exposed to 30 μM ATQ for 4 days (Table 1). However, 30 μM VE did not affect the viability of LNCaP cells over 4 days of treatment.

TABLE 1
LNCaP Growth and Viability Response
to ATQ and VE Treatment

| | Control | | 30 μM VE | | 30 μM ATQ | |
|---|---|---|---|---|---|---|
| Cell Count - | 100 | (17.1) | 103.4 | (19.7) | 42.5 | (6.2) |
| % control (SD)¹ | | | | | | |
| % Viable (SD)² | 88.8 | (3.2) | 92.0 | (1.9) | 82.9 | (4.2)³ |

¹Determined using light microscopy using a hemacytometer.
²Determined using propidium iodide exclusion and flow cytometry.
³P < 0.05 compared to control cells.

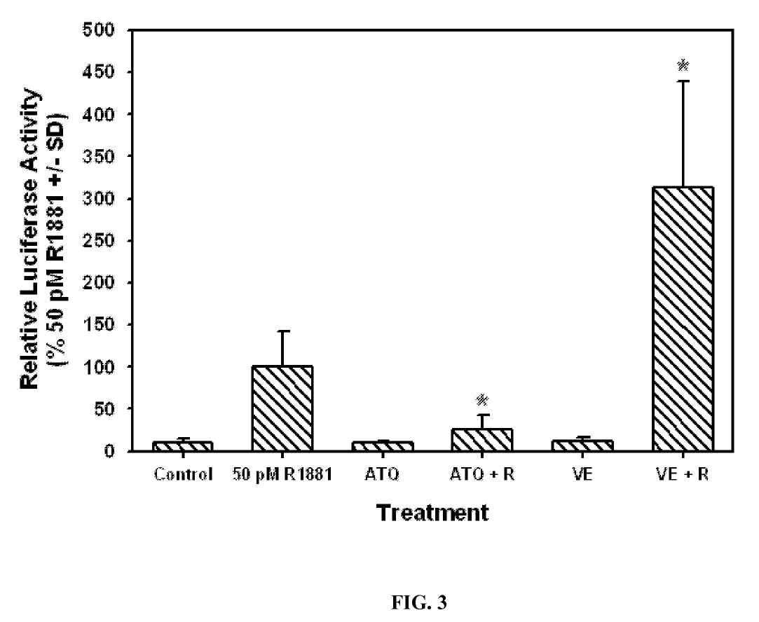

Modulation of androgen-induced promoter activation by ATQ and VE treatment: Androgen-sensitive reporter vectors are stimulated in LNCaP cells exposed to the synthetic androgen R1881. Modulation of a luciferase reporter vector driven by the androgen-sensitive MMTV promoter was assessed in LNCAP cells after treatment with 30 μM ATQ and VE (FIG. 3). ATQ was found to inhibit R1881-induced MMTV activity after 2 days (FIG. 3).

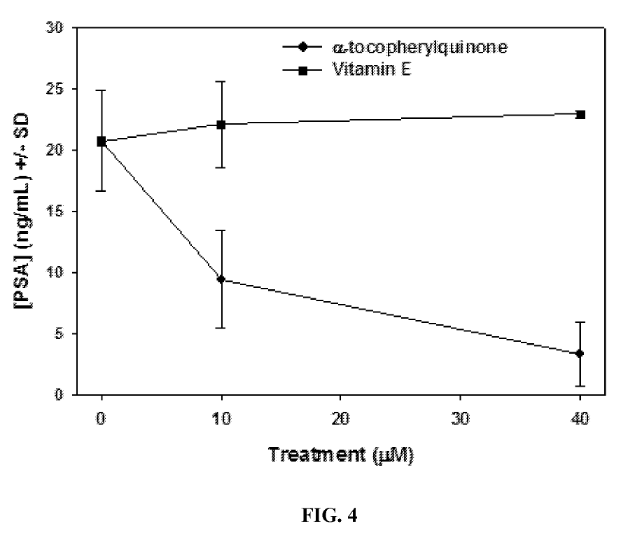

Inhibition of PSA release by ATQ treatment from LNCaP cells. In addition to androgen-sensitive promoter activation, androgens also stimulate the release of PSA from LNCaP cells. LNCaP cells treated with 10 μM ATQ showed a 50% reduction in R1881-induced PSA release compared to untreated control cells (FIG. 4). Furthermore, treatment with 40 μM ATQ produced a 4-fold decrease in PSA release compared to untreated controls (FIG. 4). In contrast, treatment with 10 to 40 μM VE did not affect androgen-induced PSA release from LNCaP cells (FIG. 4).

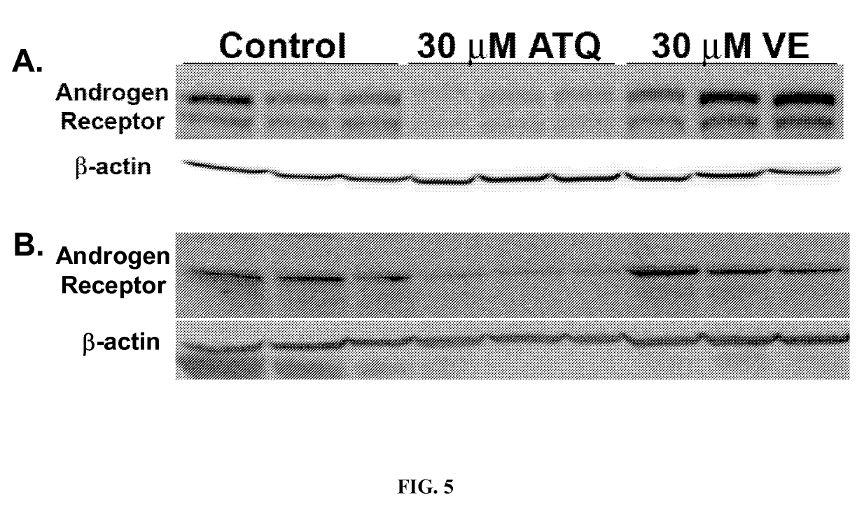

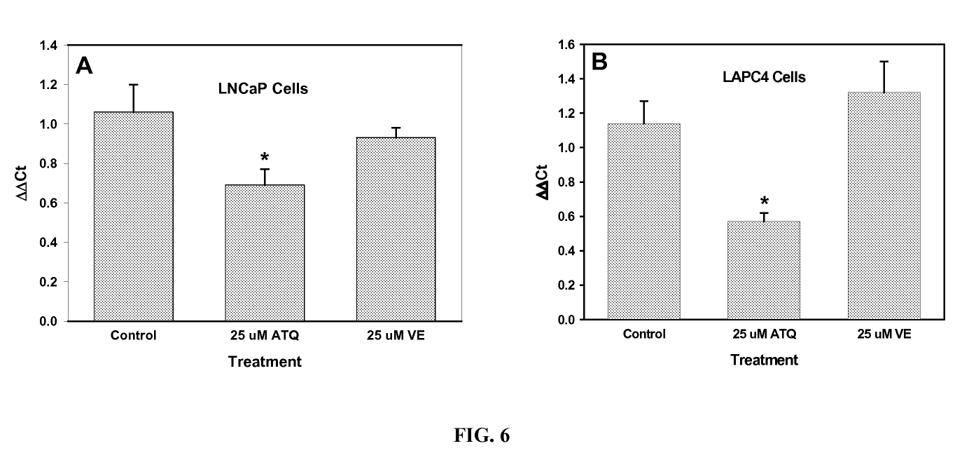

Decreased AR protein and AR mRNA levels in LNCaP and LAPC4 cells treated with ATQ: To determine if the effects on androgen-stimulated responses in LNCaP cells may result from effects on the AR, AR protein and mRNA levels were determined after ATQ treatment for 4 days. A 4-fold reduction in AR protein levels were observed in LNCaP cells treated with 30 μM ATQ and LAPC4 cells treated with 25 μM ATQ, as determined by immunoblot analysis (FIG. 5). AR protein levels were slightly increased by VE treatment (FIG. 5). AR mRNA levels were decreased 2-fold after treatment with 30 μM ATQ in both LNCaP and LAPC4 androgen-sensitive human prostate cancer cells (FIG. 6).

Although the invention has been described in connection with specific embodiments, it is understood that the invention is not limited to such specific embodiments but encompasses all such modifications and variations apparent to a skilled artisan that fall within the scope of the appended claims.

We claim:

1. A method for preventing or treating an androgen-dependent disease in a mammal, the method comprising the step of:
administering to a mammal in need thereof a pharmaceutically or prophylactically effective amount of a compound or a pharmaceutically acceptable salt or solvate thereof wherein the compound is defined by formula I: 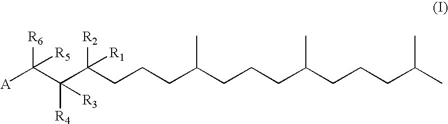
wherein A is 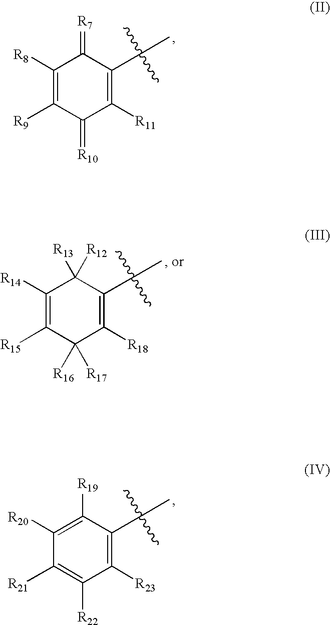
wherein R₁-R₆, R₈, R₉, and R₁₁-R₂₃ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₂ group, an alkylamino group defined as an —NHR group wherein R is an alkyl group having 1-3 hydrocarbons, a dialkylamino group defined as a —NRR′ group wherein R and R′ are alkyl groups having 1-3 hydrocarbons, an amido group defined as an —CONH₂ group, an alkylamido group defined as an —CONHR group wherein R is an alkyl group having 1-3 hydrocarbons, dialkylamido group defined as a —CONRR′ group wherein R and R′ are alkyl groups having 1-3 hydrocarbons, a nitro group defined as a —NO₂ group, a nitrile group defined as a —CN group, an acetate group defined as an —OAc group, a carbonyl group defined as a —CHO or —COR group wherein R is an alkyl group having 1-3 hydrocarbons, a carboxyl group defined as a —COOH group, an vinyl group defined as a —CH═CH₂ group, an ethinyl group defined as an —C≡CH group, a sulfonic acid group defined as a —SO₃H group, a sulfonamide group defined as a —SO₂NH₃ group, an alkylsulfonyl group defined as an —SO₂R group wherein R is an alkyl group having 1-3 hydrocarbons, a thio group defined as a —SH group, or a thioalkyl group defined as a —SR group wherein R is an alkyl group having 1-3 hydrocarbons, and
wherein R₇ and R₁₀ are identical or different and represent a methylene group defined as a ═CH₂ group, an ethylene group defined as an ═CHCH₃ group, an n-propylene group defined as a ═CHCH₂CH₃ group, an isopropylene group defined as an ═C(CH₃)₂ group, an imine group defined as an ═NH group, a thione group defined as a ═S bonded S, or an “one” group defined as an ═O bonded O.

2. The method of claim 1, wherein A is

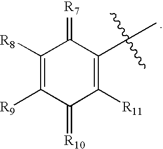

3. The method of claim 2, wherein R₇ and R₁₀ are identical or different and represent an imine group defined as an ═NH group, a thione group defined as a ═S bonded S, or an “one” group defined as an ═O bonded O.

4. The method of claim 1, wherein A is III.

5. The method of claim 4, wherein R₁₂, R₁₃, R₁₆, and R₁₇ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₂ group, an alkylamino group defined as an —NHR group wherein R is an alkyl group having 1-3 hydrocarbons, a dialkylamino group defined as a —NRR′ group wherein R and R′ are alkyl groups having 1-3 hydrocarbons, a nitro group defined as a —NO₂ group, a nitrile group defined as a —CN group, a carbonyl group defined as a —CHO or —COR group wherein R is an alkyl group having 1-3 hydrocarbons, a carboxyl group defined as a —COOH group, an vinyl group defined as a —CH═CH₂ group, an ethinyl group defined as an —C≡CH group, a sulfonic acid group defined as a —SO₃H group, an alkylsulfonyl group defined as an —SO₂R group wherein R is an alkyl group having 1-3 hydrocarbons, a thio group defined as a —SH group, or a thioalkyl group defined as a —SR group wherein R is an alkyl group having 1-3 hydrocarbons.

6. The method of claim 1, wherein A is IV.

7. The method of claim 6, wherein R₁₉, and R₂₂ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₁₂ group, an alkylamino group defined as an —N₁₁R group wherein R is an alkyl group having 1-3 hydrocarbons, a thio group defined as a —SH group, or a thioalkyl group defined as a —SR group wherein R is an alkyl group having 1-3 hydrocarbons.

8. The method of claim 1, wherein A is structure II or IV, wherein R₁ and R₂ are identical or different and represent a hydrogen defined as a —H atom, a methyl group defined as a —CH₃ group, or a hydroxyl group defined as a —OH group, wherein R₃, R₄, R₅, and R₆ are identical or different and represent a hydrogen defined as a —H atom or a methyl group defined as a —CH₃ group, wherein each of R₈, R₉, R₁₁, R₂₀, R₂₁, and R₂₃ is a methyl group defined as a —CH₃ group, wherein R₇ and R₁₀ are identical or different and represent a thione group defined as a ═S bonded S or an “one” group defined as an ═O bonded O, and wherein R₁₉ and R₂₂ are identical or different and represent a hydroxyl group defined as a —OH group or a thio group defined as a —SH group.

9. The method of claim 8, wherein the compound is α-tocopherylquinone.

10. The method of claim 8, wherein the compound is α-tocopherylhydroquinone.

11. The method of claim 1, wherein the mammal is a human.

12. The method of claim 1, wherein the disease is selected from prostate cancer, benign prostatic hyperplasia, prostatic intraepithelial neoplasia, androgenic alopecia, hirsutism, acne, seborrhea, adrenal hyperplasia, precocious puberty, and polycystic ovary syndrome.

13. The method of claim 1, wherein the method is for treating an androgen-dependent disease in the mammal.

14. The method of claim 1, wherein the method is for preventing prostate cancer.

15. The method of claim 1, wherein the method is for treating prostate cancer.

16. The method of claim 1, wherein the method is for preventing benign prostatic hyperplasia.

17. The method of claim 1, wherein the method is for treating benign prostatic hyperplasia.

18. The method of claim 1, wherein the method is for preventing or treating androgenic alopecia.

19. A method for inhibiting the proliferation of androgen-dependent prostate tumor cells comprising the step of exposing said tumor cells to a compound or a pharmaceutically acceptable salt or solvate thereof in an amount effective to inhibit the proliferation of the tumor cells wherein the compound is defined by formula I: 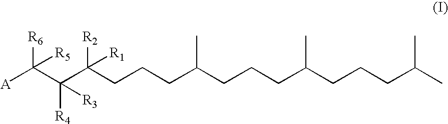
wherein A is 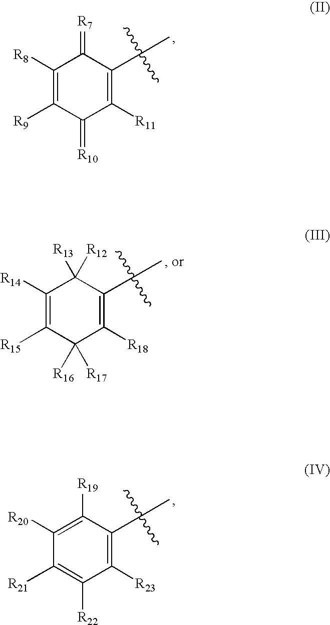
wherein R₁-R₆, R₈, R₉, and R₁₁-R₂₃ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₂ group, an alkylamino group defined as an —NHR group wherein R is an alkyl group having 1-3 hydrocarbons, a dialkylamino group defined as a —NRR′ group wherein R and R′ are alkyl groups having 1-3 hydrocarbons, an amido group defined as an —CONH₂ group, an alkylamido group defined as an —CONHR group wherein R is an alkyl group having 1-3 hydrocarbons, dialkylamido group defined as a —CONRR′ group wherein R and R′ are alkyl groups having 1-3 hydrocarbons, a nitro group defined as a —NO₂ group, a nitrile group defined as a —CN group, an acetate group defined as an —OAc group, a carbonyl group defined as a —CHO or —COR group wherein R is an alkyl group having 1-3 hydrocarbons, a carboxyl group defined as a —COOH group, an vinyl group defined as a —CH═CH₂ group, an ethinyl group defined as an —C≡CH group, a sulfonic acid group defined as a —SO₃H group, a sulfonamide group defined as a —SO₂NH₃ group, an alkylsulfonyl group defined as an —SO₂R group wherein R is an alkyl group having 1-3 hydrocarbons, a thio group defined as a —SH group, or a thioalkyl group defined as a —SR group wherein R is an alkyl group having 1-3 hydrocarbons, and
wherein R₇ and R₁₀ are identical or different and represent a methylene group defined as a ═CH₂ group, an ethylene group defined as an ═CHCH₃ group, an n-propylene group defined as a ═CHCH₂CH₃ group, an isopropylene group defined as an ═C(CH₃)₂ group, an imine group defined as an ═NH group, a thione group defined as a ═S bonded S, or an “one” group defined as an ═O bonded O.

20. A pharmaceutical composition comprising:
a compound or a pharmaceutically acceptable salt or solvate thereof wherein the compound is defined by the formula: 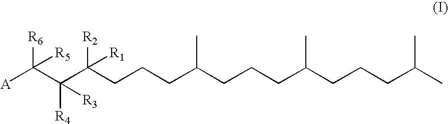
wherein A is 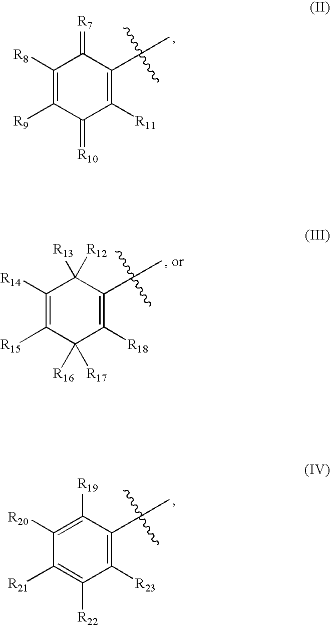
wherein R₁-R₆, R₈, R₉, and R₁₁-R₂₃ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₂ group, an alkylamino group defined as an —NHR group wherein R is an alkyl group having 1-3 hydrocarbons, a dialkylamino group defined as a —NRR′ group wherein R and R′ are alkyl groups having 1-3 hydrocarbons, an amido group defined as an —CONH₂ group, an alkylamido group defined as an —CONHR group wherein R is an alkyl group having 1-3 hydrocarbons, dialkylamido group defined as a —CONRR′ group wherein R and R′ are alkyl groups having 1-3 hydrocarbons, a nitro group defined as a —NO₂ group, a nitrile group defined as a —CN group, an acetate group defined as an —OAc group, a carbonyl group defined as a —CHO or —COR group wherein R is an alkyl group having 1-3 hydrocarbons, a carboxyl group defined as a —COOH group, an vinyl group defined as a —CH═CH₂ group, an ethinyl group defined as an —C≡CH group, a sulfonic acid group defined as a —SO₃H group, a sulfonamide group defined as a —SO₂NH₃ group, an alkylsulfonyl group defined as an —SO₂R group wherein R is an alkyl group having 1-3 hydrocarbons, a thio group defined as a —SH group, or a thioalkyl group defined as a —SR group wherein R is an alkyl group having 1-3 hydrocarbons, and
wherein R₇ and R₁₀ are identical or different and represent a methylene group defined as a ═CH₂ group, an ethylene group defined as an ═CHCH₃ group, an n-propylene group defined as a ═CHCH₂CH₃ group, an isopropylene group defined as an ═C(CH₃)₂ group, an imine group defined as an ═NH group, a thione group defined as a ═S bonded S, or an “one” group defined as an ═O bonded O; and
a pharmaceutically acceptable carrier,
with the proviso that the compound is not α-tocopherylquinone or α-tocopherylhydroquinone.

21. A method for providing nutraceutical benefits to a mammal, the method comprising the step of:
administering to the mammal a nutraceutical composition that comprises a compound or a pharmaceutically acceptable salt or solvate thereof wherein the compound is defined by formula I: 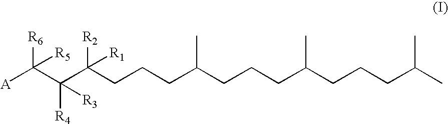
wherein A is 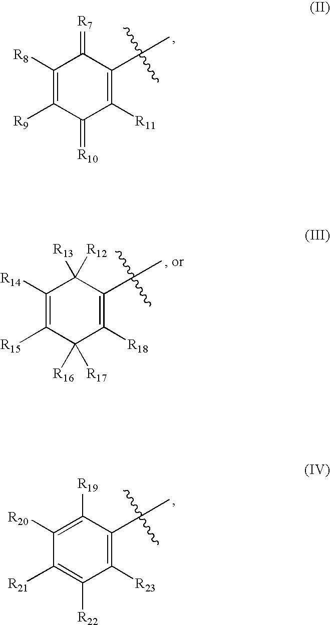
wherein R₁-R₆, R₈, R₉, and R₁₁-R₂₃ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₂ group, an alkylamino group defined as an —NHR group wherein R is an alkyl group having 1-3 hydrocarbons, a dialkylamino group defined as a —NRR′ group wherein R and R′ are alkyl groups having 1-3 hydrocarbons, an amido group defined as an —CONH₂ group, an alkylamido group defined as an —CONHR group wherein R is an alkyl group having 1-3 hydrocarbons, dialkylamido group defined as a —CONRR′ group wherein R and R′ are alkyl groups having 1-3 hydrocarbons, a nitro group defined as a —NO₂ group, a nitrile group defined as a —CN group, an acetate group defined as an —OAc group, a carbonyl group defined as a —CHO or —COR group wherein R is an alkyl group having 1-3 hydrocarbons, a carboxyl group defined as a —COOH group, an vinyl group defined as a —CH═CH₂ group, an ethinyl group defined as an —C≡CF group, a sulfonic acid group defined as a —SO₃H group, a sulfonamide group defined as a —SO₂NH₃ group, an alkylsulfonyl group defined as an —SO₂R group wherein R is an alkyl group having 1-3 hydrocarbons, a thio group defined as a —SH group, or a thioalkyl group defined as a —SR group wherein R is an alkyl group having 1-3 hydrocarbons, and
wherein R₇ and R₁₀ are identical or different and represent a methylene group defined as a ═CH₂ group, an ethylene group defined as an ═CHCH₃ group, an n-propylene group defined as a ═CHCH₂CH₃ group, an isopropylene group defined as an ═C(CH₃)₂ group, an imine group defined as an ═NH group, a thione group defined as a ═S bonded S, or an “one” group defined as an ═O bonded O.

22. The method of claim 21, wherein the compound is α-tocopherylquinone.

23. The method of claim 21, wherein the compound is α-tocopherylhydroquinone.

24. The method of claim 21, wherein the mammal is a human.

25. A nutraceutical composition comprising:
a compound or a pharmaceutically acceptable salt or solvate thereof wherein the compound is defined by formula I: 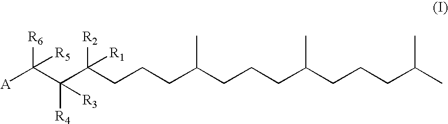
wherein A is 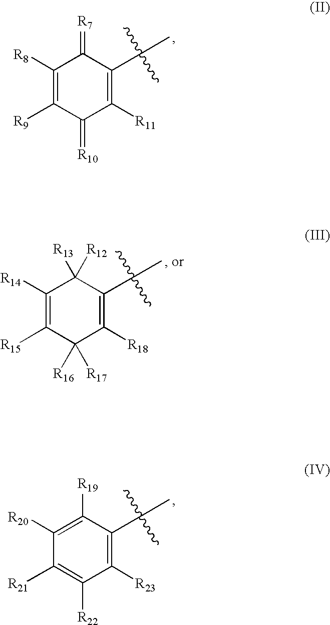
wherein R₁-R₆, R₈, R₉, and R₁₁-R₂₃ are identical or different and represent a hydrogen defined as a —H atom, a halogen selected from a —F atom, a —Cl atom, a —Br atom, and an —I atom, a hydroxyl group defined as a —OH group, an alkyl group having 1-3 hydrocarbons, an alkoxy group defined as an —OR group wherein R is an alkyl group having 1-3 hydrocarbons, an amino group defined as an —NH₂ group, an alkylamino group defined as an —NHR group wherein R is an alkyl group having 1-3 hydrocarbons, a dialkylamino group defined as a —NRR′ group wherein R and R′ are alkyl groups having 1-3 hydrocarbons, an amido group defined as an —CONH₂ group, an alkylamido group defined as an —CONHR group wherein R is an alkyl group having 1-3 hydrocarbons, dialkylamido group defined as a —CONRR′ group wherein R and R′ are alkyl groups having 1-3 hydrocarbons, a nitro group defined as a —NO₂ group, a nitrile group defined as a —CN group, an acetate group defined as an —OAc group, a carbonyl group defined as a —CHO or —COR group wherein R is an alkyl group having 1-3 hydrocarbons, a carboxyl group defined as a —COOH group, an vinyl group defined as a —CH═CH₂ group, an ethinyl group defined as an —C≡CH group, a sulfonic acid group defined as a —SO₃H group, a sulfonamide group defined as a —SO₂NH₃ group, an alkylsulfonyl group defined as an —SO₂R group wherein R is an alkyl group having 1-3 hydrocarbons, a thio group defined as a —SH group, or a thioalkyl group defined as a —SR group wherein R is an alkyl group having 1-3 hydrocarbons, and
wherein R₇ and R₁₀ are identical or different and represent a methylene group defined as a ═CH₂ group, an ethylene group defined as an ═CHCH₃ group, an n-propylene group defined as a ═CHCH₂CH₃ group, an isopropylene group defined as an ═C(CH₃)₂ group, an imine group defined as an ═NH group, a thione group defined as a ═S bonded S, or an “one” group defined as an ═O bonded O; and
an acceptable carrier,
with the proviso that the compound is not α-tocopherylquinone or α-tocopherylhydroquinone.
